(19) United States
(12) Patent Application Publication
(10) Pub. No.: US 2008/0015527 A1
(43) Pub. Date: Jan. 17, 2008

(54) Catheter assemblies having protective sheaths

(76) Inventor: Jamie Glen House, Colorado Springs, CO (US)

Correspondence Address: MOAZZAM & ASSOCIATES, LLC, 7601 LEWINSVILLE ROAD, SUITE 304, MCLEAN, VA, 22102, US

(21) Appl. No.: 11/487,486

(22) Filed: Jul. 17, 2006

(51) Int. Cl. A61M 1/00

(52) U.S. Cl. 604/326

(57) ABSTRACT

Devices and methods are disclosed for a catheterization process, particularly useful for self-performed catheterizations. A catheter is enclosed in a sheath which has a textured surface. This textured surface provides grip that is particularly useful for those with low manual dexterity.

### BACKGROUND OF THE INVENTION

1. Field of the Invention

The present invention relates generally to catheter assemblies. More particularly, the present invention relates to catheter assemblies having protective sheaths.

2. Background of the Invention

It has become relatively commonplace for the occasional, intermittent or periodic catheterization of an individual's urinary bladder to be employed, as opposed to placement and maintenance of an indwelling catheter that continuously drains urine from the bladder. Short-term or repeated catheterization is appropriate, or even required, for many persons who are in a hospital setting, a nursing home, doctor's office, rehabilitation facility, or in the home. For example, a patient is sometimes catheterized to treat such conditions as urinary retention, the inability to evacuate urine, or for the purpose of obtaining a sterile urine specimen from a patient in a doctor's office.

The need for intermittent catheterization of an individual sometimes arises due to problems typically associated with long-term use of indwelling catheters, such as infections, urethral damage, and bladder damage. Long-term use of an indwelling catheter is also a risk factor for bladder cancer. This is often the case for persons having a neurogenic urinary condition, such as in a spinal cord injury, multiple sclerosis, stroke, trauma, or other brain injury. Conditions that interfere with the individual's ability to voluntarily void the bladder may also arise post-surgically or as a result of benign prostatic hypertrophy or diabetes. Many of the affected individuals are capable of, and would prefer to perform self-catheterization. For many, the level of risk and discomfort of repeated catheterizations carried out over the course of a day (at 3-6 hour intervals, for example) are offset by the accompanying convenience, privacy, or self-reliance that is achieved. Some of the major difficulties that arise in self-catheterization are the lack of satisfactory catheterization kits, the problem of maintaining the required level of sanitation during the procedure, and the difficulty of sometimes performing the procedure under conditions of restricted space and privacy.

In assisted, or non self-catheterizations, it is common practice in hospitals to employ a catheterization tray, which typically includes a sterile drape, gloves, a conventional catheter, antiseptic solution, five cotton balls, lubricant, forceps, underpad, and a urine collection container. Assisted catheterization is usually performed with the patient in a supine position. Maintaining a sterile field during the procedure can still be a problem, however, and the “cath tray” procedure is impractical for use with some individuals and situations today.

Many individuals with spinal cord injuries or other neurological diseases routinely perform intermittent catheterization several times a day using conventional catheters or kits and the “clean technique.” Clean technique means that the urethral area is initially swabbed with antiseptic, and efforts are made to avoid contamination of the catheter during the procedure. The user's hands are not sterile and a sterile field is not maintained. Clean technique is used instead of sterile technique, generally, for two reasons. First, it is very difficult, if not impossible, for individuals who are performing self-catheterization to adhere strictly to sterile technique. Secondly, these individuals are required to catheterize themselves between 3 and 6 times a day, and the cost of a new sterile catheter and the accessories required to perform sterile catheterization becomes excessively expensive for some users. Sometimes an individual will reuse a “cleaned” catheter. As a result, the use of “clean technique” will many times result in contamination and subsequent infection of the urinary tract, causing significant morbidity and cost to the patient and society.

Besides using a sterile technique and the clean technique, another method used to maintain sterility in catheter use is to surround the catheter with a sheath. The user then holds the catheter through the sheath while pushing the catheter through into the urethra, avoiding direct contact with the catheter before insertion. The sheath provides some initial protection against contamination of a catheter. However, conventional sheaths may not be adapted for use by those with limited manual dexterity. In some cases the sheaths can become slippery if exposed to moisture or wetness. In hospitals, nurses are accustomed to using a glove on a bare catheter, which makes it very easy to manipulate the catheter. This advantage can be lessened with sheathed catheters.

Thus, there is a need in the healthcare industry for a catheter assembly that enables all users, particularly those users with limited dexterity, to grip the catheter assembly and manipulate it with decreased difficulty and increased dexterity as compared to conventional catheter assemblies currently on the market. Further, the catheter assembly should be easy to use and control, while having minimal parts that interfere with the catherization process.

### SUMMARY OF THE INVENTION

The present invention addresses the shortcomings of conventional catheter assemblies by providing a catheter assembly with a sheath with qualities that provide increased gripping ability for all users, particularly those with limited manual dexterity. A textured surface is employed in the sheath that gives these users enough gripping ability to easily hold the sheath with their hand(s) while pulling the catheter through. The sheath will remain in-between the user's hand and the catheter during insertion preventing direct contact with the catheter. The sheath is also impervious to body fluids, atmospheric air, and microorganisms, which ensures sterility after manufacture. This invention can be utilized in all catheter assembly systems where increased gripping ability is useful, including but not limited to, indwelling and intermittent catheter systems, as well as in-patient and out-patient designs.

Furthermore, the present invention can include components that assist in the catheterization process. Particular exemplary embodiments include lubrication so the catheter can slide smoothly down the urinary tract, and a guiding tip, which may also have a lubricant reservoir, to give the user something solid to line up the catheter to the urethra. Also, a hydrophilic coating may be used on the catheter of certain embodiments to hold the lubricant onto the catheter while in the urinary tract.

In one particular exemplary embodiment of the present invention, a sheath is disclosed which prevents slippage of the fingers in every direction. The sheath includes a surface with protruding ribs cross-woven in a hatch pattern. These ribs increase the friction between the surface of the sheath and the user's fingers, thereby decreasing the likelihood of slippage.

In another exemplary embodiment of the present invention, a sheath is disclosed which not only prevents slippage, but also has increased protrusions for an even greater amount of friction. The sheath includes a surface with a series of protruding mounds. These mounds can be spaced apart so that the user's fingers fit comfortably in between the mounds, thereby assuring the user that minimal slippage will occur.

### BRIEF DESCRIPTION OF THE DRAWINGS

FIG. 1A shows an external perspective view of a catheter surrounded by a sheath according to an exemplary embodiment of the invention.

FIG. 1B shows an external perspective view of a catheter surrounded by a sheath according to an exemplary embodiment of the invention with the sheath pulled back to expose part of the catheter.

FIG. 1C shows an external perspective view of a catheter surrounded by a sheath attached to a guide tip at the proximal end according to an exemplary embodiment of the present invention.

FIGS. 2A-2G show close-up views of a surface texture of a sheath according to various exemplary embodiments of the present invention.

FIG. 2H shows an external perspective view of a catheter surrounded by a sheath with a box showing the section exploded in FIGS. 2A-2G.

FIG. 3 shows a perspective view of a guiding tip according to an exemplary embodiment of the present invention.

### DETAILED DESCRIPTION OF THE INVENTION

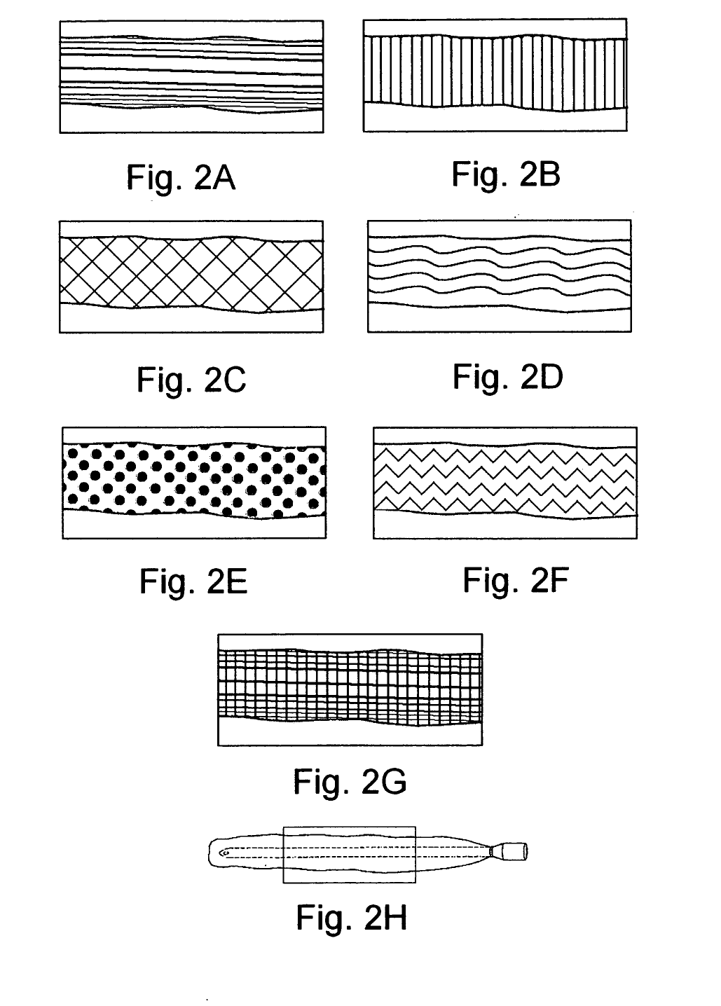

The present invention is beneficial for all users of urinary catheter assemblies by allowing an easier gripping surface for the assembly. In particular, the present invention includes devices and methods for urinary catheterization for patients who want to self-catheterize in a sterile and safe manner, yet do not have the manual dexterity to complete the process efficiently. In order to achieve the level of sterility required to avoid infection, a sheath 100 is used to cover the portion 180 of the catheter that is insertable into the urethra. The sheath 100 can cover the catheter 110 from time of storage until full insertion. The user can pull the catheter 110 through the sheath 100 without touching the catheter 110 itself. Since this task can be particularly difficult for those with low manual dexterity, the present invention employs a textured surface FIG. 2 on the outside of the sheath 100 to aid in gripping, handling and manipulating of the sheath, and hence, the catheter itself.

This texture is essentially any topography of a non-uniform elevation. Uniform elevation would be any surface that is flat and smooth, which increases the likelihood that the surface is either slippery or can be made slippery quickly by contact with moisture, either on the user's hand or elsewhere. The present invention seeks to employ surfaces that have ridges, bumps, notches or any other type of substantially non-flat or non-smooth surface to enhance the contact and grip with a user's hand and fingers. There are many possible textures for the sheath 100. These textures can be placed on the inside of the sheath as well, providing a user with grip on the catheter. FIG. 2A shows a series of ribs running parallel with the surface of the sheath 100, which is the basic technique for providing grip, preventing slipping perpendicular to the movement of the catheter 110. If used on the inside of the catheter, the parallel ribs will give the user enough grip to manipulate the catheter while still allowing it to slide forward through the sheath. FIG. 2B shows a series of ribs running perpendicular to the surface of the sheath 100, which prevents slipping against the movement of the catheter 110. It also allows the sheath to fold up more easily as it compresses towards the proximal end during insertion. FIG. 2C shows a series of ribs interwoven in a hatch pattern, which prevents slipping in any direction. FIG. 2G shows another embodiment of a hatch patter. FIG. 2D shows a series of wavy ribs running longitudinal with the surface of the sheath 100, which performs the functions of the pattern in FIG. 2A, but with an added ability to prevent movement in the transverse direction. FIG. 2E shows a series of bumps or projections covering the surface of the sheath 100, which provides the user with larger obtrusions for even more grip. If the surface of the sheath is wet, these bumps will serve as dry pads for the fingers while the moisture rests in the valleys in between. FIG. 2F shows a series of ribs in a tread-like pattern, much like the bottom of a pair of running shoes or a tire, which is another technique of preventing slipping in any direction. Many of these patterns have ribs or ridges of the like. These ribs or ridges should stand high enough to prevent slipping it the given direction, but not so high as to interfere with the user's ability to move the catheter through the center or the sheath's ability to collapse to the proximal end during insertion.

The material of the sheath 100 should either be manufactured in a sterile manner or be able to withstand sterilization after manufacture. Materials suitable for this use include, but are not limited to, polyethylene, polyvinyl chloride, high density polyethylene, polypropylene, polytetrafluoroethylene, silicon oils, other synthetic organic polymers, and the like. The sheath 100 material should be manufactured in a way that leaves the sheath 100 with a non-smooth finish.

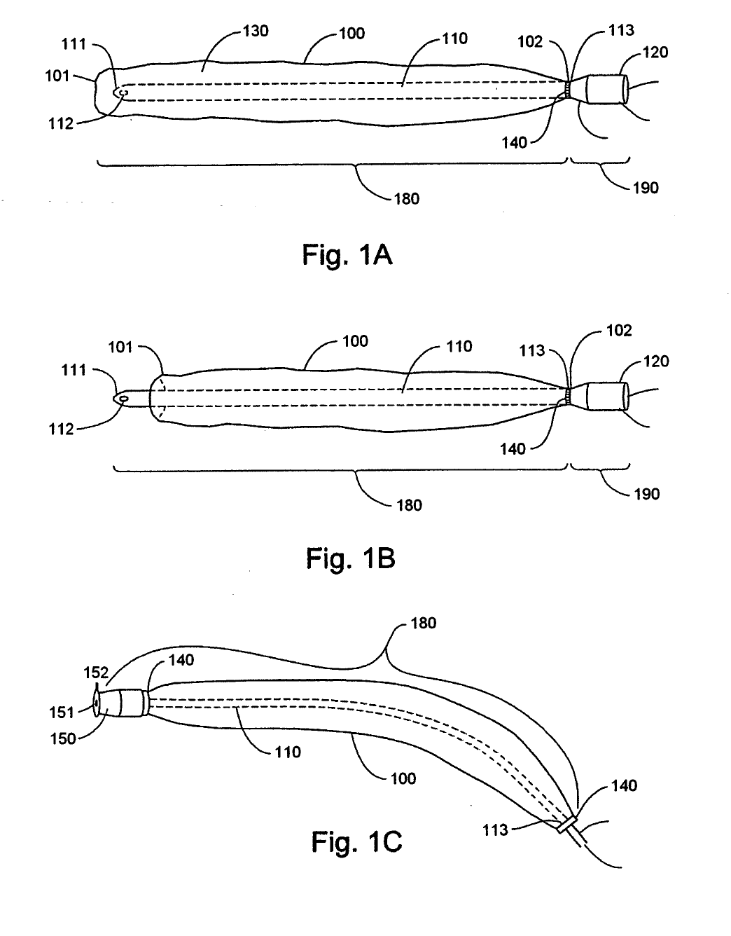

An exemplary embodiment of the present invention using a textured sheath is shown in FIG. 1. The proximal end of the sheath 101 surrounds the proximal tip of the catheter 111 and is closed at the end. If there is a guiding tip 150 present at the proximal end of the catheter 111, the sheath 100 will be attached to it with plastic or elastomeric ties or bands 140, or by heat-sealing.

The distal end of the sheath 102 is attached near the distal end of the urethra insertable portion of the catheter 180 with plastic or elastomeric ties or bands 140, or by heat-sealing. Alternately, the distal end of the sheath 102 could be attached to the outlet 120 if the catheter employs one. This outlet 120 could then be used to attach a urine bag or the like.

In order to perform a catheterization using this device the user should first open the proximal end of the sheath 100, exposing the proximal end of the catheter 111, as shown in FIG. 1 B. The user then holds the proximal tip of the catheter 111 with the sheath 100 between the user's hand and the catheter 110 and pulls the sheath 100 with the other hand. As the user pulls the sheath 100, which is attached to the catheter at its distal end 113, the catheter 110 will be pushed through the sheath 100 and into the urethra, causing the sheath 100 to bunch up at the proximal end 101. The textured surface FIG. 2 will allow the user to easily pull the sheath 100, forcing the catheter 110 into the urethra. For embodiment's that utilize a guiding tip 150 at the proximal end of the catheter 111, a catheterization can be performed without opening the proximal end of the sheath 101. The user can simply push the catheter 110 through the guiding tip 150 and into the urethra.

This process continues until the catheter 110 runs all the way through the urethra and into the bladder. Once inside, fluid from the bladder will stream into the catheter through the opening 112 at the proximal tip of the catheter 111. Provided the catheter distal end 113 is lower in altitude than the catheter proximal end 111, fluid will flow through the catheter, out the outlet 120, and into a receptacle. When the bladder has been drained of all fluid the catheter 110 is then pulled out of the urethra by the user, and disposed.

In preferred embodiments, the sheath 100 will be filled with enough lubricant 130 to coat the insertable length of the catheter 180. This will be a water-based lubricant of the type used on rectal thermometers and enemas such as KY-JELLY®. As the catheter 110 is pushed through the sheath 100 the lubricant 130 is pushed through as well, lubricating the insertable portion of the catheter 180 on its way into the urethra. The lubricant 130 will ease the process of sliding the catheter 110 into the urethra by reducing the friction between the catheter 110 and the urethra. By reducing the friction the user can insert the catheter 110 faster and with less pain.

In another embodiment as shown in FIG. 1C, the catheter 110 can also have a guiding tip 150 at the proximal end of the catheter 111. The guiding tip 150 helps the user hold the catheter 110 in place while inserting it into the urethra. The guiding tip 150 has a throughbore 151 in the center which the catheter 110 can slide through.

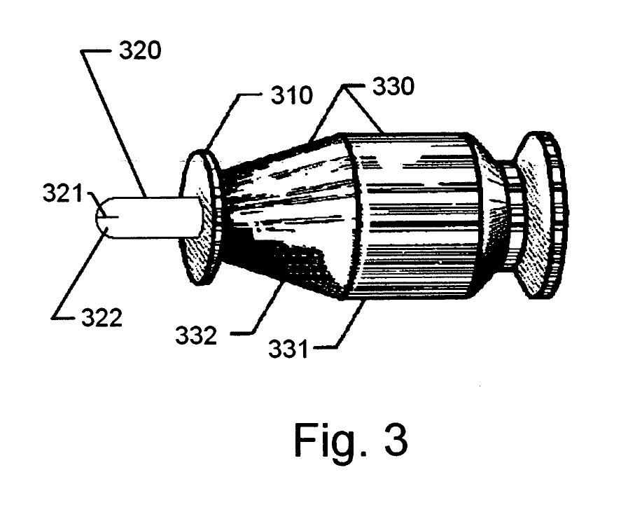

An exemplary embodiment of the guiding tip 150 is illustrated in FIG. 3. At the proximal end of the guiding tip is a collar or flange 310, having a diameter between 15 and 25 mm, which, during insertion, rests on the outside of the urethra. At the proximal end of the collar 310 is a short tube 320 just wider than the catheter 110. This tube 320 ends in a rounded top with two cuts in the top 321. When the catheter 110 is pushed through the top the tube 320 splits into multiple tabs 322, allowing the catheter 110 to pass therethrough. Towards the distal end of the guiding tip 150 there is also a reservoir portion 330. The distal portion of the reservoir includes a hollow cylinder 331 while the proximal half has a hollow frustoconical section 332. The reservoir portion 330 can contain the same lubricant 130 that is held inside the sheath 100. This makes the guiding tip 150 longer and bulkier, and gives the user more surface area to hold onto while sliding the catheter 110 through. The reservoir portion 330 can be formed by a variety of ways, including, for example, that shown by U.S. Pat. No. 6,090,075, entitled “Disposable Urinary Catheterization Assembly,” issued on Jul. 18, 2000, and hereby incorporated by reference herein in its entirety. On the outside of the reservoir 330, a texture can be added for extra gripping. This texture can be the same texture FIG. 2 as used on the sheath 100, but any texture other than a smooth surface will add grip.

In other embodiments, the catheter 110 will be coated with a hydrophilic substance, commonly known as HYDROGEL. This hydrophilic coating helps the catheter 110 to hold the lubricant 130 on its surface while inside the urinary tract. One such hydrophilic substance that can be used is agarose, known also under its trade name BIOGEL A.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A catheter assembly comprising:
a catheter;
a sheath, having a textured surface, surrounding and enclosing at least a portion of the catheter.

2. The catheter assembly of claim 1, wherein the textured surface includes ridges that are longitudinal to a long axis of the sheath.

3. The catheter assembly of claim 1, wherein the textured surface includes ridges that are perpendicular to a long axis of the sheath.

4. The catheter assembly of claim 1, wherein the textured surface includes ridges that are in a diamond shape.

5. The catheter assembly of claim 1, wherein the textured surface includes ridges that are in a wave shape.

6. The catheter assembly of claim 1, wherein the textured surface includes bumps.

7. The catheter assembly of claim 1, wherein the textured surface includes ridges that are in a tread pattern.

8. A urinary catheter assembly comprising:
a sterile, flexible catheter; and
a sheath, having a non-smooth surface, surrounding and enclosing at least a portion of the catheter.

9. The urinary catheter assembly of claim 8, wherein the textured surface includes ridges that are longitudinal to a long axis of the sheath.

10. The urinary catheter assembly of claim 8, wherein the textured surface includes ridges that are perpendicular to a long axis of the sheath.

11. The urinary catheter assembly of claim 8, wherein the textured surface includes ridges that are in a diamond shape.

12. The urinary catheter assembly of claim 8, wherein the textured surface includes ridges that are in a wave shape.

13. The urinary catheter assembly of claim 8, wherein the textured surface includes bumps.

14. The urinary catheter assembly of claim 8, wherein the textured surface includes ridges that are in a tread pattern.

15. An intermittent urinary catheter assembly comprising:
a flexible catheter;
a sheath, having a textured surface, surrounding and enclosing at least an insertable portion of the catheter.

16. The intermittent urinary catheter assembly of claim 15, wherein the textured surface includes ridges that are longitudinal to a long axis of the sheath.

17. The intermittent urinary catheter assembly of claim 15, wherein the textured surface includes ridges that are perpendicular to a long axis of the sheath.

18. The intermittent urinary catheter assembly of claim 15, wherein the textured surface includes ridges that are in a diamond shape.

19. The intermittent urinary catheter assembly of claim 15, wherein the textured surface includes ridges that are in a wave shape.

20. The intermittent urinary catheter assembly of claim 15, wherein the textured surface includes bumps.

21. The intermittent urinary catheter assembly of claim 15, wherein the textured surface includes ridges that are in a tread pattern.

22. A method of using a catheter with a textured sheath comprising the steps of:
Opening the sheath at its proximal end, exposing the proximal end of the catheter;
Holding the catheter proximal end by grabbing sheath around it so that the user's fingers do not contact the catheter, and lining it up with the urethra;
Pulling the sheath with the other hand towards the proximal end, forcing the catheter to slide through the sheath and into the urethra, and continuing until catheter has reached the bladder.

23. The method of using a catheter with a textured sheath as in claim 22 where the catheter has a guiding tip there is no need to open the sheath.
